(19) United States
(12) Patent Application Publication
(10) Pub. No.: US 2023/0138192 A1
(43) Pub. Date: May 4, 2023

(54) MEDICAL SERVICE ROBOT DEVICE, AND METHOD AND SYSTEM THEREOF

(71) Applicant: Xiamen Brana Design Co., Ltd., Xiamen, Fujian Province (CN)

(72) Inventor: Zhongtang WANG, Xiamen (CN)

(21) Appl. No.: 17/911,648

(22) PCT Filed: Feb. 26, 2021
(86) PCT No.: PCT/CN2021/078035

§ 371 Date: Sep. 14, 2022

(30) Foreign Application Priority Data
Mar. 14, 2020 (CN) 202010217182.3
Jun. 7, 2020 (CN) 202010533573.6
Sep. 26, 2020 (CN) 202011082382.9
Feb. 26, 2021 (CN) PCT/CN2021/078035

(51) Int. Cl. G16H 40/20

(52) U.S. Cl. CPC G16H 40/20

(57) ABSTRACT

Disclosed are a medical service robot device and a method and system thereof. The medical service robot device comprises a general practitioner robot, a medical first-aid robot and a medical ward-round attendant robot, which respectively implement routine diagnosis and treatment, medical first-aid and medical ward-round operation by means of artificial intelligent medical techniques at an outpatient end, an emergency end and a ward end of a medical service system, thereby replacing or partially replacing doctors in the medical service system to serve human health.

### BACKGROUND OF THE PRESENT INVENTION

### Field of Invention

The invention relates to a field of a medical robot, and more particularly to devices, methods and systems for a medical service robot.

### Description of Related Arts

Most countries in the world have entered or are entering an aging society, resulting in a rapid increase in a demand for a medical service and an apparent shortage of a medical staff. In particular, when severe acute respiratory syndrome (SARS), corona virus disease 2019 (COVID-19) and other epidemics broke out, the medical staff had direct contact with a highly infectious patient, worked intensively with a decreased immunity, resulting in a nosocomial infection and even death, thereby weakening a social medical care capacity.

A medical service robot can participate in an epidemic prevention and control without cross-infection, and provide a “7 days×24 hours” service in an intelligent automatic manner. In addition, the medical service robot can be replicated and mass-produced, thus having a natural advantage in the medical services.

However, the medical services provided by an existing medical disinfection robot, a medical care robot, a surgical robot, an artificial intelligence (AI) doctor and/or an intelligent rescue vehicle cannot replace a physical medical service system due to a lower level of intelligence, a fewer treatment measure, and an imperfect diagnosis and treatment equipment.

In view of the above-mentioned deficiencies, a new type of medical service robot equipment is urgently needed. With a help of a support system, the new type of medical service robot can sense a self-state and an environmental change in real time, respond to a medical need in a timely manner, and use a remote control or an intelligent control to monitor a vital sign, perform a medical examination, and complete an examinations of physical, laboratory, pathological, etc., have a life support, manage a surgical condition, have a medical ward round, and execute a physician order, thereby replacing or partially replacing the physical health care system.

### SUMMARY OF THE PRESENT INVENTION

### Technical Problem

An objective of the present invention is to provide a medical service robot, which can perceive a self-state and an environmental change in real time, respond to a medical need in a timely manner, use a remote control or intelligent control method, monitor a vital sign, perform a medical examination, and complete a physical, laboratory, pathological and other inspection, provide a life support, complete a surgical treatment, accompany a medical ward round, execute a doctor and/or nurse order, and implement an efficient of diagnosis and treatment operation.

### Technical Solutions of Conventional Problems

### Technical Solutions

A medical service robot device according to an embodiment of the present invention comprises a general practitioner robot, a medical emergency robot, and a medical ward round accompanying robot, which are respectively set at an outpatient department, an emergency department, and an inpatient department of a medical service system.

Preferably, the outpatient department of the medical service system includes a medical outpatient clinic, an individual clinic, a health examination center, and/or an online and offline combined medical workstation. The emergency department of the medical service system includes a hospital emergency center, a hospital emergency room, and/or an emergency first aid workstation. The inpatient department of the medical service system includes a hospital inpatient ward, a medical outpatient observation room, and/or a family ward.

Preferably, the general practitioner robot comprises a chassis and a robot body, the robot body comprises a display, a mechanical arm, a mechanical neck and a main structure, the main structure comprises a medical care module, a specimen collection module, a specimen detection module, a physical examination module, a daily care module, a central data processing module, a biosafety module, a data security module, and a power supply module, the chassis carries the robot body, and the mechanical neck connects the display and the main structure.

Preferably, the medical emergency robot comprises a chassis, a mechanical arm and a main structure, the chassis comprises a wheeled intelligent mobile structure, or a quadruped intelligent mobile structure, or a crawler-type intelligent mobile structure, the main structure comprises a state perception module, an environment perception module, a human-machine interaction module, a wireless communication module, a central data storage and processing module, a physical inspection module, a laboratory inspection module, a life support module, a surgical emergency module, a disinfection module, an intelligent storage module, a warning equipment, a universal bracket, and a display.

Preferably, the medical ward round accompanying robot comprises a chassis and a robot body, the robot body comprises a power supply module, a wireless communication module, an equipment module, a medicine device module, a cleaning and disinfection module, a printing and copying module, a data storage and processing module, and a human-machine collaborative module.

Preferably, the chassis comprises a plurality of detection device, the detection device at least comprises a camera, an ultrasonic radar, a lidar, a gyroscope, a 3D sensor, and an infrared sensor.

Preferably, the mechanical arm comprises a left nine-axis mechanical arm and a right nine-axis mechanical arm, which are respectively connected to an upper left end of the main structure and an upper right end of the main structure. The mechanical arm comprises a gripping part, a suction cup part and an auxiliary diagnosis part.

Preferably, the display comprises a touch screen, a detection device, a state perception module, a biometric identification module, an interactive module, a wireless communication module, and an air quality monitoring module.

Preferably, the medical care module comprises a first box, the first box comprises a blood pressure measurement instrument, a blood oxygen saturation measurement instrument, a pulse measurement instrument, a body temperature measurement instrument, and a grip strength measurement instrument, which is arranged on an outer wall of the main structure and connected with an intelligent lead screw lifting instrument.

Preferably, the specimen collection module comprises a finger blood collection instrument, a venous blood collection instrument, a pleural and ascites collection instrument, a urine collection instrument, a stool collection instrument, and a plurality of universal collection instrument, which are arranged on an upper part of the main structure. The specimen detection module is arranged inside the main structure, which comprises a blood detection instrument, an excrement detection instrument, a urine detection instrument, a stool detection instrument, a pleural effusion and ascites detection instrument, an antigen antibody detection instrument, and a nucleic acid detection instrument.

Preferably, the physical inspection module comprises an electrocardiogram instrument, an electroencephalogram instrument, an X-ray inspection instrument, an ultrasound inspection instrument, and a respiratory function inspection instrument, which share the display and an intelligent cloud diagnosis workstation.

Preferably, the biological safety module comprises an internal disinfection instrument, an external disinfection instrument, an air purification instrument, and a waste treatment instrument.

Preferably, the life support module comprises an oxygen generator, a negative pressure suction device, an endotracheal intubation device, a non-invasive ventilator, a defibrillator, a pacemaker, an automatic cardiopulmonary resuscitation machine, an emergency medicine, an infusion equipment, and an infusion pump; the surgical emergency module comprises an external fixation splint, a plaster bandage, a tourniquet, a sterile dressing material, an ice pack, a disinfectant, and a folding stretcher, which are stored in an intelligent cabinet.

Preferably, the intelligent storage module comprises a small item storage part and a large item storage part, the small item storage part comprises a plurality of storage box, an induction conveyor belt, an induction door, a tray, and a plurality of storage cell. The storage box is arranged on both sides of the induction conveyor belt, and divided into at least two layers and four areas, each area comprises a plurality of storage cell, each storage cell is provided with a runner instrument. The large item storage part comprises a plurality of chamber, the chamber comprises an induction hatch, a transmission board, a controller, a second micromotor, and a lead screw instrument.

A method of a medical service robot according to an embodiment of the present invention comprises a general practitioner service method, a medical emergency service method, and a medical ward round accompanying service method, which are respectively implemented in an outpatient unit, an emergency unit, and an inpatient unit of a medical service system.

Preferably, the general practitioner service method 100 comprises the following steps: 110: deployment of a general practitioner robot; 120: the general practitioner robot arriving at a predetermined service position; 130: identifying a user by means of a biometric identification module; 140: using a camera, an interactive module to communicate with the user, and implementing a medical history collection, a body examination and a medical ward round; 150: implementing a physical examination, collecting a specimen for a laboratory examination; 160: executing a doctor order according to a program algorithm; 170: integrating a data of the medical history, the physical examination, the laboratory examination, the doctor order and a execution of the doctor order to form into a data set, and generate a medical and nursing medical record document; 180: completing a diagnosis and treatment operation.

Preferably, the medical emergency service method comprises a plurality of step 200 as follow: 210: deploying a plurality of functional module of a medical emergency robot according to a task; 220: self-inspection, if finding an abnormality, timely debugging and maintaining equipment, and supplementing a plurality of drug and consumable; 230: standby at a predetermined location, or having an area cruise, monitoring an environmental information; 240: obtaining an information, responding to a need; 250: implementing a rescue; 260: having a self-check again, supplementing a consumption, cleaning and disinfecting; and 270: being in service continuously.

Preferably, the medical ward round accompanying service method comprises a plurality of step 300 as follow: 310: deploying a medical ward round accompanying robot; 320: having a human-machine collaborative training; 330: preparation before a medical ward round; 340: the medical ward round accompanying robot implementing a medical ward round with an interactive collaboration; 350: medical ward round accompanying robot continuing the medical ward round to a next patient; 360: medical ward round accompanying robot having the interactive collaboration after the medical ward round; and 370: medical ward round accompanying robot standing by for a next medical ward round.

A medical service system according to an embodiment of the present invention comprises a plurality of software, a plurality of algorithm, a cloud server, a 5G network, an intelligent medical system, a medical data encryption system, a diagnosis and treatment data recording system, a human-machine fusion protocol, a map navigation system, an update and supplement of pharmaceutical consumables and equipment system, a call transfer system, a logistics transportation system, an intelligent access control system, and a fee settlement system configured to collaborate a general practitioner robot, a medical emergency robot and a medical ward round accompanying robot replacing a doctor and/or nurse to perform a medical service in a medical service system.

### Beneficial Effects of the Invention

### Beneficial Effects

(1) The above-mentioned medical service robots can perform an outpatient diagnosis and treatment, an emergency rescue and a medical ward round in the outpatient, emergency and ward of a medical service system in an autonomous or human-machine collaborative manner. Furthermore, according to a task requirement, a plurality of functional module of the above-mentioned medical service robots can be increased, decreased or randomly combined. The above-mentioned medical service robots can work 24 hours a day without sleep, and replace or partially replace most of a doctor and/or nurse work in the medical service system, thus alleviating a relative shortage of the doctor and/or nurse labor resource.

(2) The general practitioner robot integrates an intelligent mobile chassis and a plurality of diagnosis and treatment function module, so as to perform a medical examination, a ward round, a physical inspection, a sample collection, and a laboratory test by means of a human-machine interaction and/or a human-machine fusion technology, independently or remotely. With a help of a smart medical system, the general practitioner robot can diagnose, issue a doctor order, perform a routine treatment, generate a medical record and document automatically, thereby improving medical efficiency.

(3) The general practitioner robot can cooperate with another medical robots to implement a daily patient care, a surgical operation, an emotional support, and an environmental disinfection, thereby replacing a basic diagnosis and treatment operation of a general practitioner and/or a junior specialist and reducing a medical staff labor.

(4) The mobile chassis of the above-mentioned medical emergency robot can be adapted to an intelligent mobile structure according to an application scenario to increase a traffic capacity and achieve a various service area. The mechanical arm is provided with a grasp instrument, a suction cup instrument and an auxiliary diagnosis instrument, which can assist in completing a complex operation. A plurality of function module sets on a robot body can sense a change in itself and environment in real time, obtain a drug consumable type, quantity and expiration date, and a performance status of a linked medical equipment, make a quickly check according to a medical emergency logic, share a display and an intelligent diagnosis workstation, thereby reducing equipment size and acquisition cost.

(5) A life support system and a surgical emergency facility set up in the medical emergency robot can basically complete routine medical rescue. In addition, a plurality of function module can be increased or decreased according to a task. For example, when used in an ambulance, the size of the medical emergency robot should not be too large or too heavy, the mechanical arms can be reduced; when used in a home and a community, a smart storage module, a physical inspection equipment, a laboratory inspection equipment and a surgical emergency equipment can be appropriately simplified, and the mechanical arms, the physical inspection equipment and the laboratory inspection equipment can be removed, and the types of medicines and consumables can be reduced, thus further reducing equipment size and acquisition cost.

(6) The medical emergency robot and medical ward round companying robot can perform a timely bedside laboratory inspection and a physical inspection, and automatically record a various of data in an entire diagnosis and treatment process, thus saving the consultation time of requesting a relevant professional and a professional equipment, and reducing a burden on medical staff, thereby further improving medical efficiency.

(7) The medical ward round accompanying robot can perform a ward round operation by itself or in a human-machine collaboration, assist a doctor and/or nurse to prepare for a ward round. During a medical ward round, a plurality of equipment can meet a basic diagnosis and treatment need in a timely manner, including collecting a medical ward rounds data, automatically generating a ward round log, and recreating a scene of medical ward round, assisting an execution of a doctor order, bringing a medical item to a ward or treatment room, so as to reduce a medical error, improve the efficiency of a medical service, and reduce the doctor and/or nurse labor.

### BRIEF DESCRIPTION OF THE DRAWINGS

FIG. 1 is a schematic diagram of a front view of a general practitioner robot according to an embodiment of the present invention.

FIG. 2 is a schematic diagram of a side view of a general practitioner robot according to an embodiment of the present invention.

FIG. 3 is a schematic diagram of a first box of a general practitioner robot according to an embodiment of the present invention.

FIG. 4 is a schematic diagram of a second box of a general practitioner robot according to an embodiment of the present invention.

FIG. 5 is a schematic diagram of a first box lifting device of a general practitioner robot according to an embodiment of the present invention.

FIG. 6 is a schematic diagram of a hand accessories of a nine-axis robotic arm on a right side of a general practitioner robot according to an embodiment of the present invention.

FIG. 7 is a schematic diagram of a pressure sensor matrix on a right side of a nine-axis robotic arm of a general practitioner robot according to an embodiment of the present invention.

FIG. 8 is a schematic diagram of a urine specimen collection instrument of a general practitioner robot according to an embodiment of the present invention.

FIG. 9 is a schematic diagram of a urine specimen collection instrument with a urine bag of a general practitioner robot according to an embodiment of the present invention.

FIG. 10 is a schematic diagram of a stool specimen collection instrument of a general practitioner robot according to an embodiment of the present invention.

FIG. 11 is a schematic diagram of a universal specimen collection instrument of a general practitioner robot according to an embodiment of the present invention.

FIG. 12 is a schematic diagram of a specimen detection instrument of a general practitioner robot according to an embodiment of the present invention.

FIG. 13 is a schematic diagram of an X-ray inspection instrument of a general practitioner robot according to an embodiment of the present invention.

FIG. 14 is a schematic diagram of a disinfection spray instrument of a general practitioner robot according to an embodiment of the present invention.

FIG. 15 is a schematic diagram of a front view of a medical emergency robot according to an embodiment of the present invention.

FIG. 16 is a schematic diagram of a small item storage part of a medical emergency robot according to an embodiment of the present invention.

FIG. 17 is a schematic diagram of a runner instrument of a medical emergency robot according to an embodiment of the present invention.

FIG. 18 is a schematic diagram of a longitudinal cross-sectional structure of a large item storage chamber of a medical emergency robot according to an embodiment of the present invention. and

FIG. 19 is a schematic diagram of a cross-sectional structural a large item storage chamber of a medical emergency robot according to an embodiment of the present invention.

### DETAILED DESCRIPTION OF THE PREFERRED EMBODIMENT

### Preferred Embodiments of the Present Invention

A medical service robot device in a preferred embodiment of the present invention includes a general practitioner robot, a medical emergency robot, and a medical round companion robot. The general practitioner robot is set at the outpatient end of the medical service system, and the medical emergency robot is set at the medical service system. In the emergency department, the medical rounds accompanying robots are installed on the ward side of the medical service system, using remote control or intelligent control methods to monitor human vital signs, whole body examinations, lung function, ECG, ultrasound, EEG, X-rays Wait for physical inspections, collect samples and complete laboratory and pathological tests, implement routine diagnosis and treatment, emergency care and medical rounds, automatically generate medical records, execute medical orders, emotional escort, and environmental disinfection.

### Invention Embodiments

### Embodiments of the Present Invention

A plurality of technical solutions in an embodiment of the present invention will be clearly and completely described as follow with reference to a plurality of accompanying drawing. Obviously, the embodiment is only a part of the embodiment of the present invention, rather than all the embodiments. Based on the embodiment of the present invention, all other embodiments obtained by an ordinary skill in the art without creative efforts shall fall within a protection scope of the present invention.

It should be noted that orientation terms such as up, down, left, and right in the embodiment are only a relative concept to each other or refer to a normal use state of a product, and should not be regarded as a limiting.

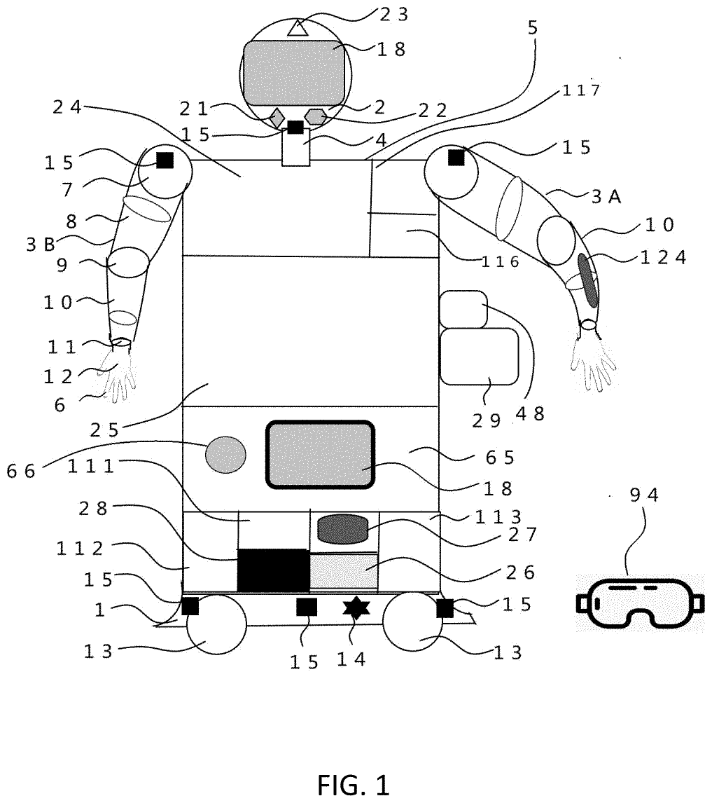

Referring to FIGS. 1-19, a medical service robot device according to a preferred embodiment of the present invention includes a general practitioner robot, a medical emergency robot, and a medical ward round companying robot. The general practitioner robot is deployed in an outpatient department of a medical service system, the medical emergency robot is deployed in the emergency department of the medical service system, and the medical ward round companying robot is deployed in an inpatient department of the medical service system, respectively. The medical service robots can be remotely controlled or intelligently controlled to monitor a human vital sign, perform a physical examination, a pulmonary function examination, an electrocardiogram (ECG) inspection, an ultrasound inspection, an electroencephalography (EEG) inspection, a X-ray inspection and another physical inspections, collect a specimen, complete a laboratory and pathological detection, and implement a diagnosis and treatment, generate automatically a medical record, execute a doctor and or nurse order, an emotional support and an environmental disinfection.

As shown in FIG. 1, a general practitioner robot according to an embodiment of the present invention comprises a chassis 1 and a robot body. The robot body comprises a display 2, a mechanical arm 3, a mechanical neck 4 and a main structure 5, the display 2 and the main structure 5 is connected by the mechanical neck 4, the mechanical arm 3 comprises a left nine-axis mechanical arm 3A and a right nine-axis mechanical arm 3B, which are respectively connected with an upper left end of the main structure 5 and an upper right end of the main structure 5, the mechanical arm 3 comprises a shoulder joint 7, an upper arm 8, an elbow joint 9, a forearm 10, a wrist joint 11, a palm 12, a hand back, a metacarpophalangeal joint, a plurality of finger 6 and a plurality of interphalangeal joint, additionally, a shaft joint is respectively set in a middle section of the upper arm 8 and the forearm 10, in order to increase the flexion flexibility of the mechanical arm 3.

The chassis 1 is a four-wheel 13 omnidirectional driving intelligent mobile carrier with a height of 15 cm-25 cm. A plurality of detection devices 15 are distributed in the chassis 1, the display 2, the mechanical arm 3, and the main structure 5 to sense an immediate change of a surrounding environment. By means of a global positioning system (GPS)/Beidou navigation system 4, an unmanned driving and/or manned remote control are available. The chassis 1 can avoid an obstacle autonomously, optimize a moving route, and reach a predetermined service location by executing a route planning algorithm based on a user instruction in a specific environment. The detection device 15 at least comprises a camera 33, an ultrasonic radar, a lidar, a gyroscope, a 3D sensor 16, and an infrared sensor 17.

It should be noted that, in response to a user need, a short-distance route, such as inside a hospital or a community or adjacent to the community, the chassis 1 can go to the predetermined service location; or the short-distance route with a complex condition, such as a step, a ravine, and/or a rough road, the chassis 1 can reach the predetermined service location with a manually assist; if the route is long, the chassis 1 can reach the predetermined service location by a vehicle, such as a car, a plane, a drone, a rail transit, or arrive at the predetermined service location by a third-party logistics system, so as to provide a door-to-door medical service.

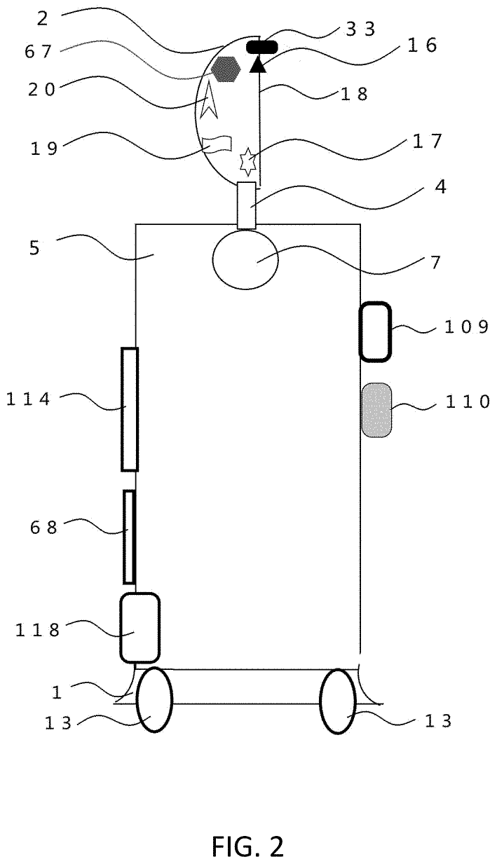

As shown in FIG. 1 and FIG. 2, a display 2 of an embodiment of the present invention comprises a touch screen 18, a detection device 15, a state perception module 19, an air quality monitoring device 20, a biometric identification module 21, an interactive module 22, a wireless communication module 23. The display 2 can be moved up and down, left and right within a certain range through a mechanical neck 4, and can also be moved to a side of a main structure 5, which is convenient for a human-machine interaction, and can also be placed horizontally for easy storage. A plurality of touch screens 18 can also be arranged on a palm 12 and a hand back of a mechanical arm 3 and a periphery of the main structure 5 to improve the human-machine interaction efficiency.

In one embodiment, an interaction module 22 can perform the human-machine interaction with a smart terminal, which comprises at least a touch screen 18. The interaction module 22 can integrate a multi-channel interaction method, including a voice, a touch screen 18, an eyesight, an expression, a iris, a palm print, a handwriting, a gait, a gesture, a lip reading, a face, a deoxyribonucleic acid (DNA), and an idea, as well as a mobile smart terminal, a smart wearable device, an implanted sensing chip in body, and groupware above information fusion, meet a usage habits of different types of a user and a communication standard of Intelligent of Things (IoT) system and equipment, and realize the human-machine interaction, or a machine-machine interaction between the devices. Of course, the present invention gradually evolves through an intelligent training, can autonomously perceive a user need, and can complete a diagnosis and treatment service without interacting with the user through a human biological characteristic.

The main structure 5 is connected with the chassis 1 in a detachable and modular manner. The main structure 5 is provided with a medical care module, a specimen collection module 24, a specimen detection module 25, an ECG inspection module, an ultrasound inspection module, an X-ray inspection module, a pulmonary function inspection module, a daily care module, a central data processing module 26, and a biological security module, a data security module 27, and a power supply module 28.

A plurality of functions of a medical care module depends on a plurality of instrument and software algorithm support configured by the display 3, the main structure 5, the mechanical arm 3, and the mechanical neck 4.

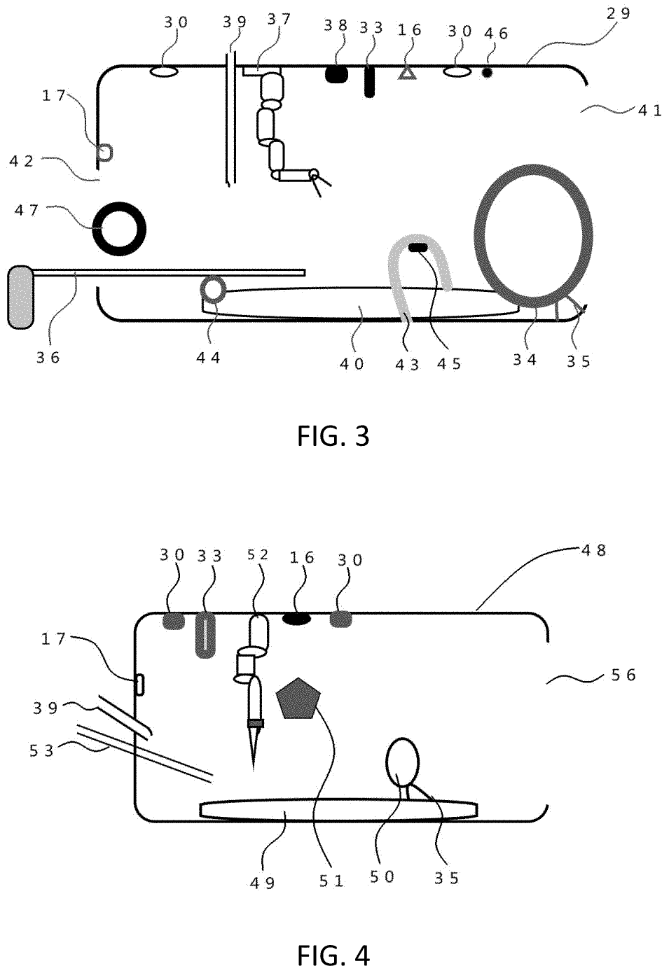

As shown in FIG. 3, a length, width, and height of a first box 29 of an embodiment of the present invention are respectively not less than 50 cm, 20 cm, and 20 cm, and a lighting 30, a camera 33, an arm strap 34, a connecting part 35, a blood collection tube with needle 36, a six-axis venous blood drawing mechanical arm 37, a 3D sensor 16, an infrared sensor 17, a blood vessel imaging device 38, a disinfectant spray device 39, an arm guard 40, an arm inlet 41, and an arm outlet 42 are arranged inside a chamber of the first box 29. A patient extends a bare upper limb from the arm inlet 41 into the first box 29, passes through the arm strap 34, and places on the arm guard 40, and the upper limb moves forward along a chute with the arm guard 40, because the connecting part 35 is fixed with an inner wall of the first box 29, so that the upper arm is restrained by a pressure, and an end portion of the upper limb that cannot be accommodated in the first box 29 protrudes from the arm outlet 42, and the disinfectant spray device 39 sprays a skin disinfectant, after the skin disinfectant is dried, according to a monitoring data of the 3D sensor 16 and the blood vessel imaging device 38, the six-axis venous blood drawing mechanical arm 37 supports the blood collection tube with needle 36, punctures the upper limb vein, and a blood specimen enters the blood collection tube with needle 36. A whole process mentioned above is under a condition of the lighting 30, the monitoring data is collected by the camera 33 and the infrared sensor 17, and the patient is prompted to cooperate with reference to an operation demonstration showing on the touch screen 18 with a help of an interaction module 22.

Moreover, a blood pressure measuring instrument 43, a blood oxygen saturation measuring instrument 44, a pulse measuring instrument 45, a body temperature measuring instrument 46, and a grip strength measuring instrument 47 are also arranged in the first box 29. With a help of the interaction module 22, referring to an operation demonstration prompt showing on the touch screen 18, the patient extends the bare upper limb from the arm inlet 41 into the first box 29, and passes through the blood pressure measuring instrument 43 and the pulse measuring instrument 45 fixed on the arm guard 40 to measure blood pressure and pulse. The body temperature measuring instrument 46 does not need touch a human body. A left hand or right hand grips the grip strength measuring instrument 47 to detect a muscle strength. The patient pinches and lifts an inner skin of the upper arm by himself, and the camera 33 and the 3D sensor 16 can collect a data to obtain a result of the patient body fat content and evaluate a nutritional status.

As shown in FIG. 4, a second box 48 of an embodiment of the present invention is provided with a finger blood collection device, with a length, width and height of a chamber of the second box 48 not less than 5 cm. The second box 48 comprises a chamber. The chamber is provided with a lighting 30, a camera 33, a 3D sensor 16, an infrared sensor 17, a finger guard 49, a finger strap 50, a connecting part 35, a puncture needle box 51, a triaxial puncture mechanical arm 52, a blood collection tube 53, and a disinfection spray device 39. With an aid of the interaction module 22, referring to a demonstration prompt showing on the touch screen 18, a patient palm is upward, and a finger is selected to extend into the second box 48 from a finger entrance 56 of the second box 48, and then the finger goes through the finger strap 50 fixed on the finger guard 49 with the connecting part 35. The finger is stretched forward, so that a base of the finger is bound, under a condition of the lighting 30, a data collected by the camera 33, the 3D sensor 16 and the infrared sensor 17 are imaged and displayed in real time on the touch screen 18, and prompt the patient to adjust the finger position to cooperate with an operation, the disinfectant spray device 39 sprays a skin disinfectant, after the skin disinfectant is dry, according to a monitoring data collected by the camera 33, the 3D sensor 16 and the infrared sensor 17, the three-axis puncture mechanical arm 52 clips a disposable puncture needle from the puncture needle box. 51, punctures a distal segment pulp of the finger, and the blood collection tube 53 collects a blood specimen for detection.

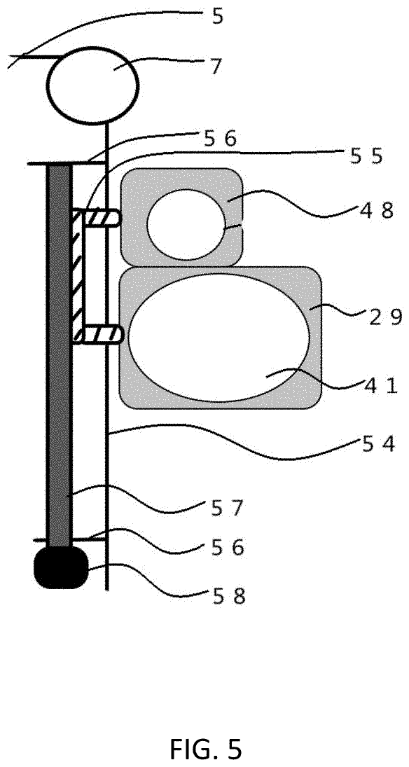

Referring to FIG. 5, the first box 29 and the second box 48 are closely connected in an embodiment of the present invention, and a composite assembly part 55 fixes the first box 29 and the second box 48 on a left outer wall 54 of the main structure 5. An intelligent lifting device consisting of a lead screw 57 and a motor 58 is arranged inside the main structure 5. The lead screw 57 and the motor 58 are fixed on a left outer wall 54 of the main structure 5 through a connecting rod 56. During an operation, the motor 58 is started, the motor 58 drives the lead screw 57 to rotate forward or reverse, and the lead screw 57 drives the composite assembly part 55 to move upward or downward, which will drive the first box 29 and the second box 48 upward or downward with a lifting range no less than 50 cm, so as to adapt to an arm height of a patient in different positions such as sitting, lying, or standing.

In addition, a camera 33, a 3D sensor 16, and an infrared sensor 17 are arranged at a front end of the display 2 according to an embodiment of the present invention to collect image data of an ups and downs of a chest and/or abdomen when the patient is in various body positions, and obtain a data of a breathing frequency and a breathing depth according to an algorithm.

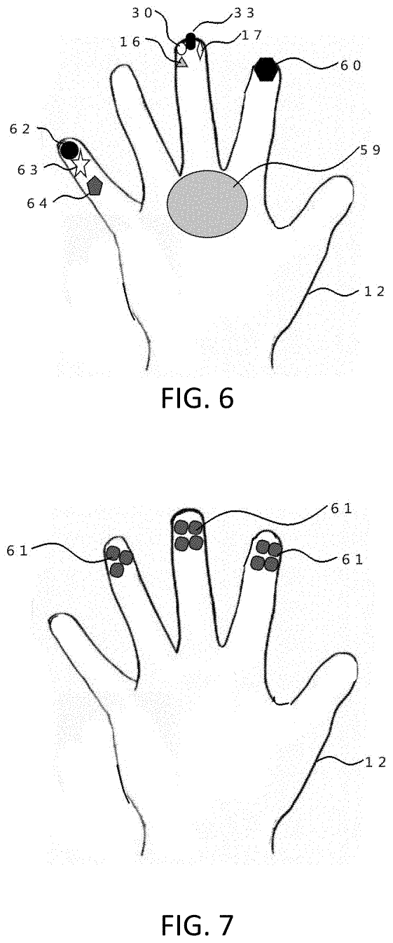

In one embodiment of the present invention, with a help of the camera 33 and the interaction module 22, referring to an operation demonstration showing on a touch screen 18, the patient is prompted to cooperate, and at least one of two modes of a doctor remote control and a robot intelligent control is used to implement a routine medical history collection, a general physical examination and a medical ward round. Referring to FIG. 6, a right nine-axis robotic arm 3B uses a stethoscope 59 set on a palm 12 and a percussion hammer 60 set at a distal end of an index finger 6 to perform an auscultation and percussion of an abdomen, a heart, and a lung. A six middle finger tip of the right nine-axis mechanical arm 3B is provided with an illumination 30, a camera 33, a 3D sensor 16, and an infrared sensor 17. A data from the camera 33, the 3D sensor 16, and the infrared sensor 17 are collected, and processed by an algorithm to sense the patient tongue image and facial image. Referring to FIG. 7, a flexible membrane pressure sensor matrix 61 is set on an abdomen of the index, middle, and ring finger 6 of the right nine-axis mechanical arm 3B to perform a whole-body palpation, a sense action potential data, and synchronously or delay mapping to an intelligent hardware of a remote physician fingertip can obtain a pulse image. In addition, the sense action potential data of the whole-body palpation collected by the flexible membrane pressure sensor matrix 61 can also be transmitted to a cloud server, and the pulse image can be obtained by a specific algorithm.

As shown in FIG. 6, an ammonia sensor 62, a hydrogen sulfide sensor 63, and an aromatic hydrocarbon sensor 64 are provided on a little finger 6 of a right nine-axis mechanical arm 3B according to an embodiment of the present invention. When the little finger 6 close to the patient body, an excrement and/or a secretion, and a local odor concentration can be detected in real time.

A doctor can use at least one of the two modes of remote control and robot intelligent control to draw up a diagnosis and treatment plan and issue a doctor order. According to a program algorithm, the left nine-axis mechanical arm 3A, the right nine-axis mechanical arm 3B, and the interaction module 22 can execute the doctor order, which includes a nursing, a diet, a project inspection, an oral medicine, an intravenous infusion medicine, a physical rehabilitation, a psychotherapy, a teleconsultation, a discharge from hospital. Furthermore, the program algorithm can integrate a medical history, a physical examination, an examination, a doctor order and a doctor order execution data to form a data set and generate a medical record document.

A daily care module of an embodiment of the present invention can mainly provide a medicine distribution, a food distribution, and a daily necessities distribution with an intelligent locker 65, and an assisting in getting up and going to a toilet, fitness and eating with a left nine-axis mechanical arm 3A and a right nine-axis mechanical arm 3B, and an emotional escort and an external communication with an interaction module 22. Referring to FIG. 1, the smart locker 65 is of an automatic pull-out design, and is arranged on a front side of a main structure 5. The smart locker 65 is divided into a thermal insulation warehouse and a low temperature warehouse, which are used for temporary storage of a medicine, a food and a daily necessity. A door of the warehouse is arranged a touch screen 18 and a smart speaker 66. The touch screen 18 can display an item in the warehouse in real time. The smart speaker 66 can broadcast a prompt information, and at the same time a user can interact with the smart speaker 66, or use a mobile smart terminal to issue a demand, call a medical staff, order a meal, and shop.

As shown in FIG. 2, an air quality monitoring device 20 provided on a rear side of a display 2 according to an embodiment of the present invention at least comprises a temperature sensor, a humidity sensor, a smoke sensor, an oxygen sensor, a carbon dioxide sensor, a carbon monoxide sensor, a particulate matter (PM) 10 sensor, and a PM 2.5 sensor for monitoring a local space environment. The air quality monitoring device 20 can control an opening and closing of a smart window, and control an operation of a room air conditioner and an air purification device 68 through a micro control module 67.

A collection and testing of human and environmental specimen is closely related to a disease diagnosis and prevention.

A specimen collection module of an embodiment of the present invention comprises a finger blood collection device disposed in the second box 48, a venous blood collection instrument disposed in the first box 29, and a urine collection instrument 69, a stool collection instrument 70, and a plurality of universal collection instrument 71 disposed on an upper part of the main structure 5 in an array. The universal collection instrument 71 is used to collect a sample of a human saliva, a nasal secretion, a pharyngeal secretion, sputum, a vomit, a prostatic fluid, a semen, a leucorrhea, a human body surface and/or an environmental object surface. A destination of the sample includes direct introduction into a testing process for analysis and detection, or temporary storage in a sample vial for inspection.

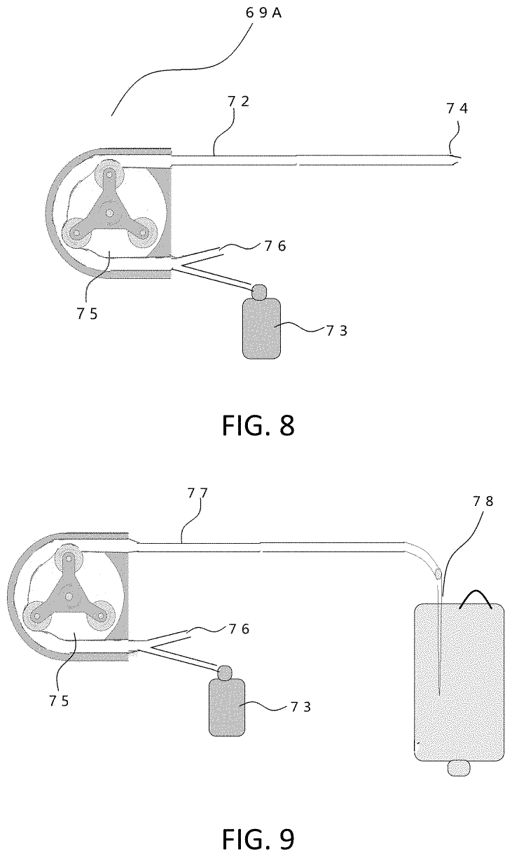

As shown in FIG. 8 and FIG. 9, a urine collection instrument 69 according to an embodiment of the present invention comprises a urinal urine collection instrument 69A and a urine bag urine collection instrument 69B, which are used to collect urine from a urinal and a urine bag, respectively. When collecting urine from the urinal, a right nine-axis mechanical arm 3B takes out a third hose 72 from an upper part of a main structure 5, and penetrates a collecting head 74 provided at an end into the urine of the urinal, then a peristaltic pump 75 to start with an intelligently control, and a urine sample enters a sample collection bottle 73 connected to a proximal end of the third hose 72 or directly enters a urine detection instrument through a liquid outlet 76. When collecting urine from the urine bag, a left nine-axis robot arm 3A and a right nine-axis robot arm 3B cooperate to take out a fourth hose 77 from the upper part of the main structure 5, and puncture into the urine of the urine bag with a needle 78 connected to an end of the fourth hose 77, the peristaltic pump 75 intelligently controlled starts, and a urine sample enters the sample collection bottle 73 connected at a proximal end through the fourth hose 77 or directly enters the urine detection instrument through the liquid outlet 76.

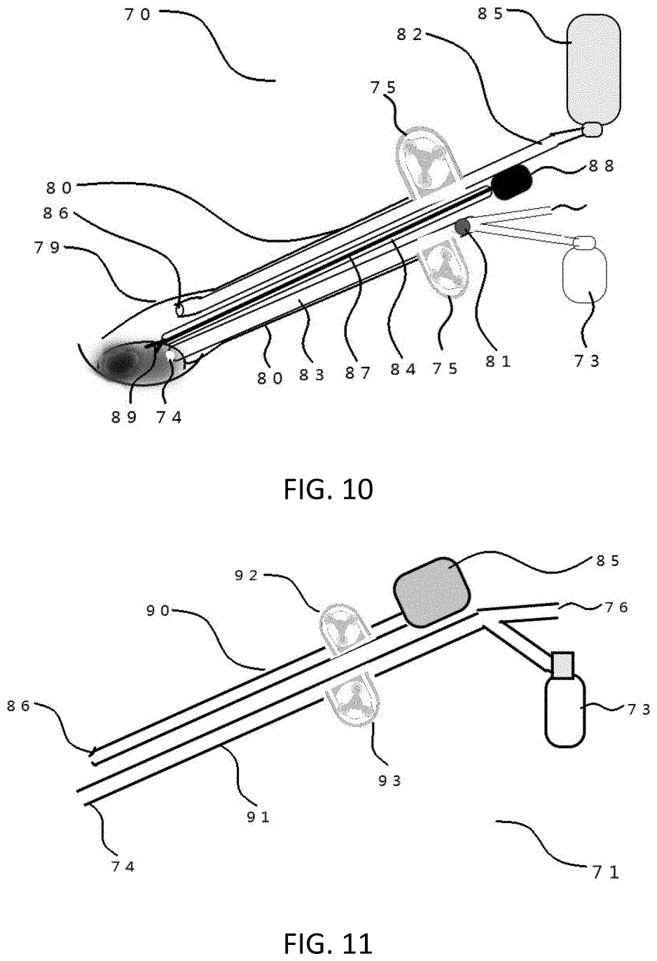

As shown in FIG. 10, a stool collection instrument 70 according to an embodiment of the present invention comprises a protective cover 79, a collection hose 80, a filter membrane 81, and a sample collection bottle 73, the protective cover 79 are arranged at a distal end of the collection hose 80. The collection hose 80 comprises a first independent hose 82, a second independent hose 83, a third independent hose 84. A proximal end of the first independent hose 82 is connected to a normal saline bag 85, a spray head 86 is arranged at a distal end, and an intelligently controlled peristaltic pump 75 is arranged between the normal saline bag 85 and the spray head 86. A proximal end of the second independent hose 83 is connected to a sample collection bottle 73, an end of the second independent hose 83 is provided with a collection head 74, and an intelligently controlled peristaltic pump 75 is arranged between the sample collection bottle 73 and the collection head 74. The third independent hose 84 has a built-in transmission shaft 87, a proximal end of the transmission shaft 87 is connected to a micromotor 88, and an end of the transmission shaft 87 is connected to a stirring head 89. In addition, a filter membrane 81 is provided at a portion of the second independent hose 83 close to the sample collection bottle 73. When collecting a stool sample, a right nine-axis mechanical arm 3B takes out the collection hose 80 from an upper part of a main structure 5, and places the protective cover 79 on a feces to prevent the feces from splashing and polluting an environment, and activates the intelligent control peristaltic pump 75 of the first independent hose 82, and normal saline contained in the normal saline bag 85 is sprayed onto the feces through a nozzle 86, the micromotor 88 starts, and drives the stirring head 89 through the drive shaft 87 of the third independent hose 84 to stir the feces into a thin paste, then the intelligently controls the peristaltic pump 75 of the second independent hose 83 starts, a stool sample enters the second independent hose 83 through the collection head 74, and a stool filtrate formed by the filter membrane 81 enters the sample collection bottle 73 or directly enters the stool detection instrument through a liquid outlet 76.

As shown in FIG. 11, a universal collecting instrument 71 of an embodiment of the present invention is mainly used for collecting a sample such as a human body surface, an environmental object surface, an oral cavity, a throat, a sputum, a leucorrhea, and a semen. The universal collecting instrument 71 comprises a first hose 90, a second hose 91, a first intelligent peristaltic pump 92, and a second intelligent peristaltic pump 93. A proximal end of the first hose 90 is connected to a normal saline bag 85, a distal end of the first hose 90 is provided with a nozzle 86, and a first intelligent peristaltic pump 92 is arranged between the normal saline bag 85 and the nozzle 86. A proximal end of the second hose 91 is connected to a sample collection bottle 73, a collection head 74 is arranged at the end of the second hose 91, and a second intelligent peristaltic pump 93 is arranged between the sample collection bottle 73 and the collection head 74. When collecting a surface sample of an environmental object, a right nine-axis mechanical arm 3B takes out the universal collecting instrument 71 from an upper part of a main structure 5, the collecting head 74 of the second hose 91 contacts the surface of the environmental object, and the first intelligent peristaltic pump 92 is activated, and the normal saline in the normal saline bag 85 is sprayed onto the surface of the environmental object through the nozzle 86 of the first hose 90, then the second intelligent peristaltic pump 93 is activated, and the normal saline containing a surface component of the environmental object enters the sample collection bottle 73 or through a liquid outlet 76 directly enters a pre-programmed detection instrument. When collecting a throat sample, a patient uses an interaction module 22 and refers to an operation prompts on a touch screen 18. A middle finger 6 of a right nine-axis mechanical arm 3B extends into the patient mouth, presses down a tongue, and exposes a throat. A data provided by a lighting 30, a camera 33, a 3D sensor 16 and an infrared sensor 17 is used to sense an exact position of the throat. A left nine-axis mechanical arm 3A takes out the universal collection instrument 71 from an upper part of the main structure 5, and the collection head 74 of the second hose 91 contacts and stimulates the throat, then the first intelligent peristaltic pump 92 starts, normal saline in the normal saline bag 85 is sprayed to the throat through a nozzle 86 of the first hose 90, and then the second intelligent peristaltic pump 93 starts, normal saline containing the throat secretion composition enters the sample collection bottle 73 through the collection head 74 and the second hose 91 or directly enters a predetermined detection instrument through a liquid outlet 76. When collecting a fluid or semi-fluid sample such as a sputum, a leucorrhea, a semen, etc., the collection head 74 of the second hose 91 is intruded into the fluid or semi-fluid sample, then the second intelligent peristaltic pump 93 starts, the collection head 74 of the second hose 91 enters the sample collection bottle 73 or directly enters a predetermined detection instrument through the liquid outlet hole 76. The above operations are based on a type and/or characteristics of the samples to receive an instruction from a central data processing module 26 for intelligent control or remote control to execute a doctor order.

In one embodiment of the present invention, a pleural effusion and ascites fluid collection is implemented in at least one mode of a remoted control mode and an intelligent control mode. When the remoted control mode is selected, a doctor wears a smart helmet or a pair of smart glasses and/or a pair of smart gloves of an artificial reality (AR)/virtual reality (VR) device 94, controls a left nine-axis mechanical arm 3A and a right nine-axis mechanical arm 3B, and guides a patient to change body position, then under a guidance, holds a blood collection tube 53 with a puncture needle, punctures under a direct vision, extracts the pleural fluid and ascites, and introduces the pleural fluid and ascites into a pleural fluid and ascites detection instrument. When the intelligent control mode is selected, with a help of the interaction module 22, the patient changes the body position, the left nine-axis mechanical arm 3A and the right nine-axis mechanical arm 3B cooperate to perform an ultrasound-guided puncture to extract pleural effusion and ascites.

In one embodiment of the present invention, a specimen detection module 25 is disposed inside a main structure 5, which comprises a blood detection instrument, an excrement detection instrument, a urine detection instrument, an antigen-antibody detection instrument 106, and a nucleic acid detection instrument. In addition, the urine detection instrument and the blood detection instrument can be used to detect a component of a pleural effusion and/or ascites sample, and the urine detection instrument can also be used to detect a component of a stool or vomit sample.

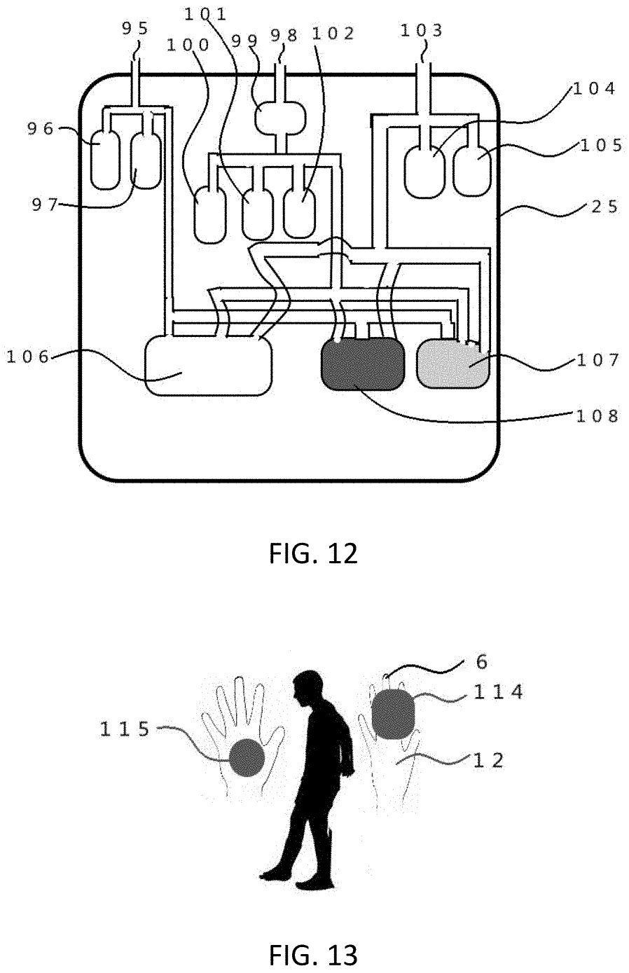

As shown in FIG. 12, a finger blood and/or venous blood sample collected in an embodiment of the present invention can enter an automatic blood cell counting and analysis equipment 96 through a first inlet 95 to obtain data on various types of blood cells, and enter an automatic biochemical analysis instrument 97 through the first entrance 95 to obtain a component data in the blood sample, which is used to analyze an organ function, an infection, an inflammation, and a tumor early warning marker. Of course, a pleural effusion sample, an ascites sample, and a cerebrospinal fluid sample can also enter the automatic blood count analysis instrument 96 and the automatic biochemical analysis instrument 97 through the first inlet 95 to detect blood cells and biochemical indicators in pleural effusion, ascites, and cerebrospinal fluid respectively.

As shown in FIG. 12, an excrement such as saliva, nasal secretion, pharyngeal secretion, sputum, vomit, prostatic fluid, semen, leucorrhea, etc. collected in an embodiment of the present invention can enter an automatic gross observation equipment 99 through a second inlet 98 to observe a basic property of the excrement, and then enters an automatic microscope observation equipment 100, an automatic smear hematoxylin-eosin (HE) staining and microscopy equipment 101, and an automatic smear immunohistochemical staining and microscopy equipment 102 to further observe a cells, a microorganism and/or other fine component of the excrement.

As shown in FIG. 12, a urine and/or stool filtrate sample collected in an embodiment of the present invention can enter a test paper detection chamber 104 and a flow analysis detection chamber 105 through a third inlet 103, then an automatic urine detection equipment and an automatic flow cytometry equipment detect and analyze the urine and/or stool filtrate sample.

The samples collected above can enter an antigen-antibody detection instrument 106 through a first inlet 95, a second inlet 98, and a third inlet 103, and use an automatic antigen-antibody detection equipment and a colloidal gold chromatographic immunoassay to qualitatively or quantitatively detect a pathogen or an antibody of the samples.

The samples collected above can enter a gene chip detection instrument 107 and a reverse transcription-polymerase chain reaction (RT-PCR) detection instrument 108 through an inlet 95, an inlet 98, and an inlet 103 for a metagene detection and a specific gene detection respectively. The gene chip detection instrument 107 comprises a gene chip storage box, an automatic sample preparation instrument, a detection kit, an automatic gene chip hybridization reaction instrument, and a signal detection instrument. The RT-PCR detection instrument 108 comprises an automatic mRNA extraction equipment, an automatic RT-PCR instrument and fluorescence detector, and a nucleic acid detection kit. The nucleic acid detection instrument is equipped with multiple sample channels, which can detect multiple samples in parallel, and detect multiple types of bacteria, viruses, and tumor-related genes in the same sample according to a program, or the same type of bacteria, viruses, and tumor-related genes in multiple samples.

In one embodiment of the present invention, when an ECG inspection is required, a right nine-axis mechanical arm 3B takes out an electrode plate and a lead wire from a first storage box 109 on a front wall of a main structure 5, and implements pasting and removing of the electrode plate on a patient body surface, selects a standard limb lead, and/or a pressurized unipolar limb lead and/or chest lead, and collects an ECG data from an ECG host 111 arranged inside the main structure 5 according to a program algorithm.

In one embodiment of the present invention, when an ultrasonic inspection is required, according to a program algorithm, a right nine-axis mechanical arm 3B takes out at least one of a convex array probe, a linear array probe or a phased array probe from a second storage box 110 on a front wall of a main body structure 5, holds the probe to detect on a surface of a patient body, and collects an ultrasonic data an internal ultrasonic instrument host 112 disposed in the main structure 5.

In one embodiment of the present invention, referring to FIG. 13, when an X-ray inspection is required, a right nine-axis mechanical arm 3B with an X-ray digital flat panel detector 114 clamped by a palm 12 and a finger 6 and a left nine-axis mechanical arm 3A with a ball tube 115 arranged on a palm 12 cooperate, an X-ray generator 113 arranged in the main structure 5 emits X-rays, penetrates a patient body, and implements the X-ray inspection according to a program algorithm. Finally, a data is collected by the X-ray digital flat panel detector 114 and send to a central data processing module 26.

In one embodiment of the present invention, when a lung function test is required, with a help of a camera 33 and a interaction module 22, a right nine-axis mechanical arm 3B takes out a mouthpiece of a function tester 116, assists a patient in blowing with the mouthpiece of the lung function tester 116, adopting a remote control mode by a doctor or an intelligent control mode. A data is collected by the lung function tester 116 and sends to a central data processing module 26 to generate a quantitative indicator of the lung function and evaluate the lung respiratory function.

In one embodiment of the present invention, when an EEG inspection is required, with a help of a camera 33 and an interaction module 22, a right nine-axis mechanical arm 3B takes out a headgear containing electrodes and an electroencephalograph 117 with a lead adopting a remote control mode by a doctor or an intelligent control mode, then a patient wears the headgear with the electrodes positioned exactly and fully contact a scalp, the electroencephalograph 117 is activated, a brainwave data is collected, and sent to a central data processing module 26 to generate a EEG quantitative indicator.

A central data processing module 26 of an embodiment of the present invention includes a data storage device and a data processing device, which can collect a clinical data, a sensor data, a detector data, a pulmonary function detection data, an EEG inspection data, an ultrasound inspection data, and a specimen laboratory detection data, publish the data to a cloud server. After a calculation and processing, the central data processing module 26 obtains a valid data, forms a data set, and completes a diagnosis and treatment operation in combination with an intelligent medical system and an artificial medical system.

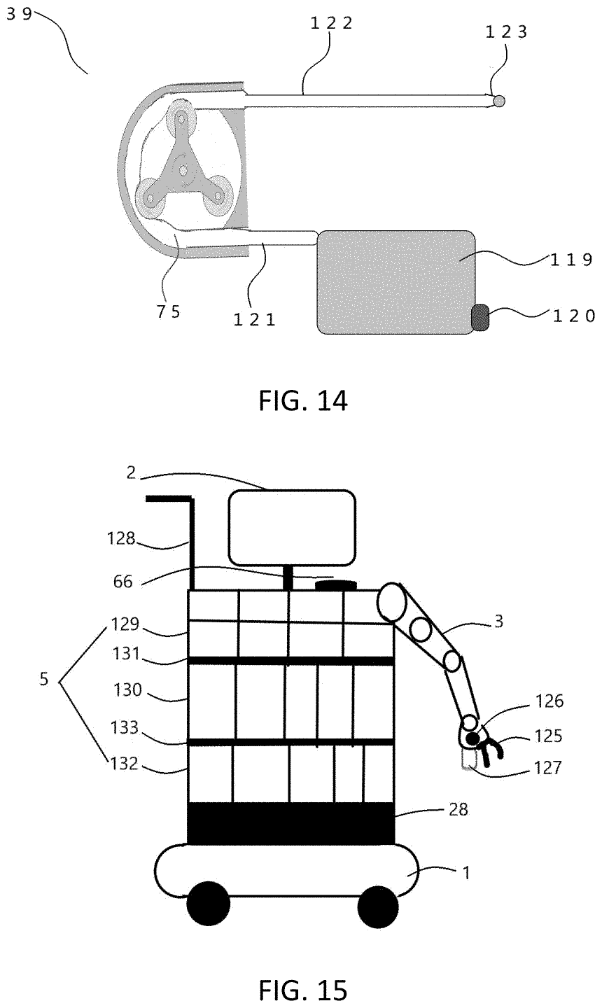

Biosecurity is of paramount importance in a medical service. The biosafety measures of an embodiment of the present invention comprise a disinfectant spray instrument 39 arranged inside of the general practitioner robot, and a disinfection instrument, an air purification instrument 68 and a waste treatment instrument 118 arranged outside of the general practitioner robot. Specifically, as shown in FIG. 14, the disinfectant spray instrument 39 comprises a storage bag 119, a filling port 120, a fifth hose 121, an intelligently controlled peristaltic pump 75, a rigid hose 122, and a spray port 123. The storage bag 119 can be filled a disinfectant through the filling port 120. When sterilizing, the intelligently controlled peristaltic pump 75 is activated, the disinfectant flows through the fifth hose 121, flows out from the rigid hose 122, and forms a high-pressure spray-like tiny droplet through the spray port 123 for sterilizing an interior space of the general practitioner robot. Moreover, the storage bag 119 can be arranged in a main structure 5, the rigid hose 122 and the spray port 123 can be arranged at an end of a middle finger 6 of a left nine-axis mechanical arm 3A used for disinfection of an outer surface of a medical equipment, a hose for specimen collection, a hospital, a home, and an isolated space. An ultraviolet lamp 124 is arranged on a forearm 10 of a left nine-axis mechanical arm 3A, and is used for ultraviolet sterilization and disinfection of an enclosed space. A waste treatment instrument 118 comprises a waste storage, a waste disinfection, and a waste disposal, which can be used to collect and harmlessly process waste from a sample, a liquid for flushing equipment, a reagent for testing, and a consumable.

In order to expand a future requirement of the general practitioner robot in an embodiment of the present invention, a specimen collection module 24 and a specimen detection module 25 can reserve a plurality of expansion slot respectively. The expansion slot can comprise an electric circuit, a water circuit, an air circuit, a sample transmission channel, a data interface, and a space, for adding a sample collection module and a detection module. Of course, one or more detection channels can also be added to the specimen detection module 25, which can realize not only a single-person or a single-sample detection, but also a single-person multi-sample detection or a multi-person multi-sample detection.

In addition, according to a user requirement, a plurality of function module of the general practitioner robot of an embodiment of the present invention can be combined as required, and a working state of the function module can also be turned off or turned on.

For example, the user can randomly select or combine one or more of a specimen collection module 24, a specimen detection module 25, a medical care module, an ECG inspection module, an ultrasound inspection module, an X-ray inspection module, a pulmonary function examination module, and a daily care module according to the requirement, in order to meet an individual needs of the user.

Furthermore, according to the user requirements, one or more specimen collection instruments in the specimen collection module 24 can be selectively reduced or increased, and one or more detection instruments in the detection module 25 can be selectively reduced or increased, so as to meet the individual needs of the user.

A user data security of an embodiment of the present invention can realize a storage and distributed operation of a user data by creating a user data center through a blockchain. The user data, a connected IoT data, a public cloud or a private cloud server connected can be completely based on a user-authorized blockchain smart contract. The user data can be written to the blockchain in real time, be released to a public or a private cloud server with a user authorization. The blockchain also realizes a security isolation of the user data, and a third-party user can be authorized to read the user data.

In one embodiment of the present invention, a data collected by a camera 33, a 3D sensor 16, an infrared sensor 17, a detector 15, a GPS/Beidou Navigation system 14 and a gyroscope scattered outside and inside of a chassis 1, a display 2, a mechanical arm 3, and/or a main structure 5 can integrate with an algorithm, and perceive a state and a surrounding environment of the general practitioner robot.

In one embodiment of the present invention, a medical service process of a general practitioner robot comprises a plurality of step 1000 as follow:

1010: deploying a general practitioner robot, including selectively combining and assembling a plurality of function module according to a user need; 1020: the general practitioner robot arriving at a predetermined service location; 1030: the general practitioner identifying a patient with a help of a biometric identification module; 1040: with a help of a camera and an interaction module, the general practitioner communicating with the patient, and performing a routine medical history collection, a physical examination and/or a medical ward round; 1050: the general practitioner performing a physical inspection, collecting a specimens for a laboratory detection; 1060: the general practitioner executing a doctor order according to a program algorithm, which can include a nursing plan, a diet plan, a plurality of examination item, a medication plan, a physical rehabilitation, a psychotherapy plan, a remote consultation, and/or a discharge; 1070: the general practitioner integrating a medical history, a physical examination, an inspection, a doctor order and/or a doctor order execution data to form a data set, and generate a medical and nursing medical record; 1080: the general practitioner completing a diagnosis and treatment operation.

As shown in FIG. 15, a medical emergency robot according to an embodiment of the present invention comprises a chassis 1, a power supply module 28, a mechanical arm 3 and a main structure 5. The chassis 1 can be adapted to a wheel-type intelligent mobile structure, a four-legged intelligent mobile structure, or a crawler-type intelligent mobile structure according to an application scenario. The mechanical arm 3 can be symmetrically arranged on both sides of the main structure 5 with a humanoid joint, and an end of the mechanical arm 3 is provided with a gripping structure 125, a suction cup structure 126 and an auxiliary diagnosis structure 127, wherein the auxiliary diagnosis structure 127 can include a visible light and far-infrared camera, a light-emitting diode (LED) Light, a 3D sensor, a smart stethoscope, a pressure sensor array, a temperature sensor, a pulse sensor, a respiration monitoring sensor, a blood pressure sensor, and a blood oxygen saturation sensor, wherein the gripping structure 125 and the suction cup structure 126 can be used for a plurality of rescue operation such as delivery of a medicine and/or an equipment, operating an equipment, carrying a stretcher, and sterilizing, wherein the suction cup structure 126 at least comprises a flexible suction end with a curved surface for suctioning a ampoule and a flat flexible suction end for suctioning another item. An uppermost part of the main structure 5 is provided with a display 2, a smart speaker 66 and a universal bracket 128. The display 2 and the smart speaker 66 have built-in a status perception module, an environment perception module, a wireless communication module, a central data storage processing module and a human-machine interaction module. The universal bracket 128 is provided with an infusion pole, an oxygen pipeline rack, a camera and a lighting equipment, an antenna equipment, a navigation sensor and a warning equipment. An upper part of the main structure 5 is provided with a small item storage part 129 and a physical inspection and testing equipment part 130. A tray 131 is set between the small item storage part 129 and the physical inspection and testing equipment part 130, which can be used as a temporary operating table. The small item storage part 129 is divided into a plurality of independent functional box, and the physical inspection and testing equipment part 130 is divided into a plurality of independent functional structure box, where a plurality of electrode plate of a ECG instrument and ECG lead, a plurality of EEG instrument and EEG lead, an X-ray digital flat panel detector and a tube and an X-ray generating instrument, an ultrasonic probe, an automatic blood cell analyzer, an automatic biochemical analyzer, an automatic urine analyzer and an automatic blood gas analyzer are respectively placed. A bulky item storage part 132 is provided at a lower part of the main structure 5. There is a folding stretcher 133 being provided between the physical inspection and testing equipment part 130 and the bulky item storage part 132.

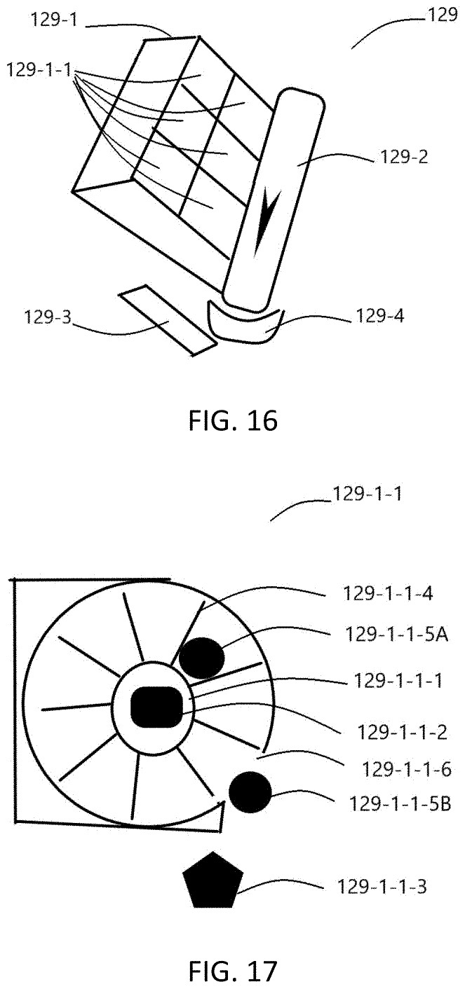

As shown in FIG. 16 and FIG. 17, a small item storage part 129 of an embodiment of the present invention can be divided into a plurality of independent functional structure area, the independent functional structure area comprises a storage compartment box 129-1, an induction conveyor belt 129-2, an induction door 129-3, a temporary storage concave plate 129-4 and a plurality of electric circuit, communication circuit and air circuit. The storage compartment box 129-1 is arranged on both sides of the induction conveyor belt 129-2, which comprises a plurality of minimum storage compartment 129-1-1, the minimum storage compartment 129-1-1 comprises a runner structure, the runner structure comprises a rotating shaft 129-1-1-1, a first micromotor 129-1-1-2, a controller 129-1-1-3, and a plurality of paddle 129-1-1-4. A width of the paddle 129-1-1-4 can be adjusted according to a size of an item to be stored configured to put a same type of small item 129-1-1-5A into the paddle 129-1-1-4 corresponding to the minimum storage compartment 129-1-1. According to an interaction with a patient for selling a drug and/or a medical care instruction, the controller 129-1-1-3 can control a rotation of the first micromotor 129-1-1-2, a small item 129-1-1-5B along with the paddle 129-1-1-4 move to an exit 129-1-1-6 of the minimum storage compartment 129-1-1. Under an action of gravity, the small item 129-1-1-5B can slide into the induction conveyor belt 129-2, then the small item 129-1-1-5B can be sent to the temporary storage concave plate 129-4. When a person or a mechanical arm 3 picks up the small item 129-1-1-5B, the induction door 129-3 can automatic open.

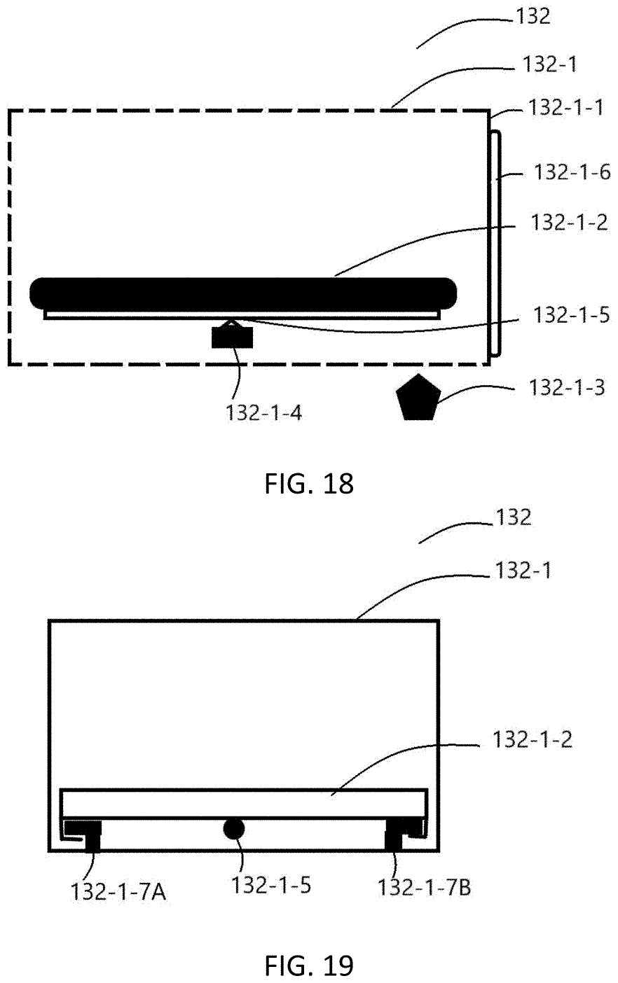

As shown in FIG. 18 and FIG. 19, a bulky item storage part 132 according to an embodiment of the present invention comprises a plurality of chamber 132-1, the chamber 132-1 can include an induction door 132-1-1, a conveying board 132-1-2, a controller 132-1-3, a micromotor 132-1-4, a lead screw structure 132-1-5, and an electric circuit, a communication line and a gas circuit. The induction cabin door 132-1-1 is opened on a surface of a shell of a main structure 5, and a secondary display 132-1-6 is arranged on a surface of the induction cabin door 132-1-1. The two sides of the conveying plate 132-1-2 are respectively connected with a sliding rail 132-1-7A and a sliding rail 132-1-7B at a lower part of the chamber 132-1. When an equipment is conveyed, the conveying plate 132-1-2 stretches out from the induction door 132-1-1, while the conveying plate 132-1-2 can also be used as a temporary solid support platform for the equipment. The equipment can include an oxygen generator, an ozone generator, a negative pressure suction instrument, an endotracheal intubation equipment and a non-invasive ventilator, a defibrillator, a pacemaker, an automatic cardiopulmonary resuscitation machine, an infusion pump, an external fixation splint, etc.

A preferred embodiment of the present invention is at a hospital emergency room for a medical emergency service.

An ordinary department of a hospital, such as an internal medicine, a surgery, a gynecology, a pediatrics, an infectious disease, and/or a radiology, must have at least an emergency room or a treatment room. Furthermore, an emergency department or an emergency center must have a plurality of emergency room.

A medical emergency robot in an emergency room of the hospital comprises a chassis 1, a mechanical arm 3 and a main structure 5. The chassis 1 can adopt a wheeled intelligent mobile structure, and the main structure 5 can be equipped with a state perception module, an environment perception module, a human-machine interaction module, a wireless communication module, a central data storage processing module, a physical inspection module, a laboratory detection module, a life support module, a surgical emergency module, a disinfection module, an intelligent storage module, a warning equipment, a universal bracket, and a display.

The medical emergency robot is on standby every day in the emergency room of the hospital, self-checks regularly, finds an abnormality, debugs and maintains equipment in time, replenishes a medicine and consumable in the intelligent storage module, replaces an expired medicine and consumable, and recharges an electricity. Once a response need is obtained, according to a call information of a patient and/or a bed number, the medical emergency robot can obstacle avoidance and migrate, timely arrival at the patient location by autonomous navigation, and identify the patient in need of a medical rescue. A routine working mode of the medical rescue operation of the medical emergency robot can include a self-rescue operation, an assisting on-site a medical staff in a rescue operation, or a man-machine coordinated rescue operation under a telemedicine control, additionally, when necessary, calling for more medical resources to participate in the rescue operation. During the rescue operation, in accordance with the medical rescue operation specifications, the medical emergency robot can perform a physical examination, access a past and recent health and medical data of the patient encrypted by a blockchain on a cloud server, and timely implement a necessary special physical inspection at a bedside, such as an X-ray inspection, an electrocardiogram inspection, an EEG inspection and/or an ultrasound inspection, and a special laboratory detection, such as a blood cell analysis, a blood gas analysis, a biochemical analysis, and/or a urinalysis, make a diagnosis and give a symptomatic treatment, automatically record the diagnosis data and the treatment data, which comprises an audio and/or a video, a body temperature data, a respiration data, a pulse data, a blood pressure data, a blood oxygen saturation data, a state of consciousness data, a facial symmetry data, a tongue movement data, a limb movement data, a skin surface damage data, a face image data, a tongue image data, a pulse image data, a physical examination and a laboratory analysis data, a human-machine interaction data, a decision basis data, a treatment process data and a follow-up process data, and automatically form an electronic rescue record that meets a medical standard, and send the data to a cloud server, then settle a medical expense, and then return to the emergency room, self-check again, supplement consumption, clean and disinfect, and continue to stand by.

A preferred embodiment of the present invention is at a crowd gathering place, such as a railway station, a terminal building, a square and a stadium for a medical emergency service.

The medical emergency robot at the crowd gathering place comprises a chassis 1, a mechanical arm 3 and a main structure 5. The chassis 1 can adopt a wheel-type intelligent mobile structure or a crawler-type intelligent mobile structure. The main structure 5 can be equipped with a state perception module, an environment perception module, a human-machine interaction module, a wireless communication module, a central data storage processing module, a physical inspection module, a laboratory detection module, a life support module, a surgical emergency module, a disinfection module, an intelligent storage part module, a warning instrument, a universal bracket and a display.

The medical emergency robot tours at the crowd gathering place, self-checks regularly, debugs and maintains equipment in a timely manner when an abnormality found, makes a supplement of medicines and consumables in the intelligent storage module, replaces a medicine and consumable that are about to expire, and recharges electricity at a specific location. Once a response need is obtained, the medical emergency robot according to a geographical location of a call information, can obstacle avoidance and migrate autonomous navigation, timely arrival at a patient location, identify the patient in need of a medical recue. A routine working mode of a rescue operation comprises adopting an autonomous rescue operation, or a man-machine coordinated rescue operation under a control of telemedicine, when necessary, calling for a support of more medical resources to participate in the rescue operation. During the rescue operation, in accordance with a medical rescue operation specification, the medical emergency robot can perform a physical examination, access to a previous health and medical data of the patient encrypted by a blockchain on a cloud server, implement a necessary special physical inspection and a laboratory detection, make a diagnosis and a symptomatic treatment. Then the medical emergency robot can record a diagnosis and treatment data automatically, and form an electronic rescue course record automatically that meets a medical standard, and send the data to the cloud server. Finally, a settlement of medical expenses, a self-inspection, a treatment of medical waste liquid waste, a supplementary consumption, an activation of ozone generator disinfection and a continued patrolling on call can be executed by the medical emergency robot.

A preferred embodiment of the present invention is in an ambulance for a medical emergency service assist.

An ambulance and a medical emergency team frequently participate in a medical rescue operation independently. They are often faced with an insufficient labor, an insufficient medicine and medical equipment, an insufficient medical skill, an opaque patient information, and a repeated laborious trip to retrieve item from a car, which restrict a further improvement of the medical emergency team.

The ambulance following-assisted medical emergency robot comprises a chassis 1, a mechanical arm 3 and a main structure 5. The chassis 1 adopts a four-legged intelligent mobile structure or a crawler-type intelligent mobile structure. The main structure 5 is equipped with a state perception module, an environment perception module, a human-machine interaction module, a wireless communication module, a central data storage processing module, a physical inspection module, a laboratory detection module, a life support module, a surgical emergency module, a disinfection module, an intelligent storage module, a warning instrument, a universal bracket and a display.

The medical emergency robot follows a medical emergency team on standby in the ambulance, self-checks regularly, debugs and maintains an equipment in a timely manner when an abnormality is found, replenishes medicines and consumables in the intelligent storage module, replaces an expired medicine and consumable, and recharges electricity. Accompany the ambulance to a patient location once a response need is obtained. A working mode when the medical emergency robot implementing a rescue comprises a plurality of step as follow: assisting a medical staff in the rescue operation or a man-machine coordinated rescue operation under a telemedicine control, during a rescue operation, assisting the medical staff in a physical examination, accessing a history health and medical data of the patient encrypted by a blockchain on a cloud server and displaying the data on the display, and assisting the medical staff to carry out a necessary special physical and laboratory detection according to an instruction of a diagnostic workstation and assisting the medical staff to make a diagnosis and a treatment symptomatically, when necessary, pulling out a tray as a temporary work plate, and a folding stretcher used to transfer the patient, in addition, during the rescue operation, according to a need of a condition and the instruction of the medical staff, a medicine and a consumable being delivered in time by the mechanical arm to the medical staff or placed on the tray, and/or an instrument being delivered from a bulky item storage part, which including an oxygen generator, a negative pressure suction instrument, an endotracheal intubation equipment and non-invasive ventilator, a defibrillator, a pacemaker, an automatic cardiopulmonary resuscitation machine, an infusion pump and/or another equipment, then the mechanical arm and an intelligent cloud diagnostic workstation assisting the medical staff to debug and select an optimal working parameter for the treatment, and deliver an external fixation splint, and/or a plaster bandage, and/or a hemostasis band, and/or a sterile dressing material, and/or an ice pack, and/or a disinfectant and/or another equipment used for a trauma treatment from the bulky item storage part, record a diagnosis and treatment data automatically, form an electronic rescue course record automatically that meets a medical standard, send the data to the cloud server, and settle a medical expense, finally the medical emergency robot returning to the ambulance, self-checking again, supplementing consumption, cleaning and disinfecting, and standing by.

A preferred embodiment of the present invention is at home and/or community for a medical emergency service.

A medical emergency robot for a family and/or a community mainly serves a common and frequently-occurring emergency patient. The medical emergency robot comprises a chassis 1, a mechanical arm 3 and a main structure 5. The chassis 1 adopts a wheeled intelligent mobile structure, and the mechanical arms 3 can be reduced from two to one or omitted. A physical inspection and laboratory analysis equipment of the main structure 5 can be simplified or omitted, thereby reducing a manufacturing cost. In addition, a user can also rent the medical emergency robot in order to further reduce a usage cost.

The medical emergency robot is on standby at home or community, self-checking regularly, debugging and maintaining equipment in a timely manner when an abnormality is found, replenishing a medicine and consumable in an intelligent storage part, replacing an expired medicine and consumable, and recharging electricity at a specific location. Once a response need is obtained, according to a geographical location of a call information, the medical emergency robot can obstacle avoidance and migrate with an autonomous navigation, timely arrive at a patient location, and identify the patient in need of a medical rescue. A working mode of a rescue operation includes an autonomous rescue operation and/or a man-machine coordinated rescue operation under a control of a telemedicine, and when necessary, a calling for a support of more medical resource to participate in a rescue operation. During a rescue operation, the medical emergency robot can conduct a physical examination in accordance with a medical rescue operation specification, access a previous health and medical data encrypted by a blockchain on a cloud server, make a diagnosis, treat a symptom, record a diagnosis and treatment data automatically, and form a medical standardized electronic rescue course record automatically, and sent the data to the cloud server. When a task is completed, the medical emergency robot can self-check, treat medical waste, supplement consumption, start ozone generator disinfection, and continue stand by.

A medical ward round accompanying robot service process 2000 according to an embodiment of the present invention can include the following steps.

2100: Deploying the medical ward round accompany robot.

According to a type and scale of a department in a hospital, one or more medical ward round accompanying robots are allocated to integrate a plurality of universal instrument and a plurality of special instrument that meet a need of a medical ward round. The special instrument can include a plurality of diagnosis and treatment instrument of the department served by the medical ward round accompanying robot, such as a plurality of physical inspection instrument, laboratory detection instrument, and treatment instrument for a specialty. In addition, a deployment of the medical ward round accompany robot also includes integrating a general operating system and a personalized programming interface, creating a map of the department, specifically marking a precise position and travel route of a bed in a ward of the department, setting up a medical ward round accompanying robot indwelling area, and establishing a regional wireless network connection.

2200: Having a human-machine collaborative training, including a plurality of step as follow:

2210: Creating a voice and a biometrics identification.

In step 2100, the deployed medical ward round companion robot completes the voice and biometric identification of all doctors, nurses, and admitted patients in the hospital, names the doctors, nurses, and admitted patients, constructs the voice and biometric identification database, and undergoes training, so that the deployed medical ward round companion robot can understand a phonetic semantics of all doctors and nurses professional and everyday words.

In addition, the voice and biometric identification database can be adjusted according to a need. For example, a new doctor or nurse in the hospital or a first-time admission patient of the hospital can be added to the database, and if a doctors or a nurse or a admission patient leave the hospital, they can be removed from the database.

In addition, a management authority for the medical ward round accompanying robot can be set by a chief doctor and/or a chief nurse of the department, which including a formulation of a rule for the usage of the medical ward round accompanying robot, so as to monitor an operation status of the medical ward round accompanying robot in real time.

2220: Creating a human-machine interaction.

The medical ward round accompanying robot deployed in step 2100 receives the human-machine interaction training from the doctor and nurse through a human-machine interaction interface. After the doctor and nurse issue a voice command, the medical ward round accompanying robot can give feedback in time, a method of the feedback includes but are not limited to retelling a content of the voice command, an execution of the voice command, the prompt when it is inconsistent with a patient medical information, and a questioning of the voice command based on a human medical knowledge map.

The medical ward round accompanying robot deployed in step 2100 receives the human-machine interaction training from a patient through the human-machine interaction interface, performs a voice or touch screen interaction, and be able to feedback a patient need during a medical ward round timely.

2300: Preparation before a medical ward round, which including a self-check, a manual confirmation, a task planning, and an execution process.

The self-check can include a medical equipment operating condition check, a material and consumable check, a medical document check, a medical equipment check. The medical equipment comprises a medical ward round accompanying robot, a physical inspection equipment, a laboratory detection equipment, a physical examination equipment, a printing and copying equipment. The material and consumable include a reagent, an electrode patch, a hand sanitizer, a disinfectant, a sanitary protection supply, a dressing and suture removal kit, a printing paper. The medical document includes an electronic medical record, an imaging examination material, and a patient informed letter.

The manual confirmation is carried out by a medical staff After the medical ward round accompanying robot submitting a self-check result, the medical staff promptly deals with an abnormal situation of the medical ward round accompanying robot, and carries out a necessary maintenance of a medical equipment, a replenishment of the materials and consumables, and an improvement of the medical documents.

The task planning can be carried out by a chief doctor and/or a chief nurse of the department. A priority can be given to arranging a large ward round and a medical team ward round in the department, and then arranging an independent doctor ward round, an anesthesiologist ward round, and a nursing ward round. In addition, a remote ward round and a consultation can be given the priority at any time. An execution process implements the medical ward round and pre-planned routes in an order of the priority, which can be adjusted according to an actual situation.

In addition, according to a need of the department, two or more medical ward round accompanying robots can be deployed to meet the need of two or more medical teams in the department to perform the medical ward rounds in parallel.

In addition, the task planning can be sent to a smart mobile terminal, and/or a desktop office terminal, and/or a voice broadcast device of the medical staff participating in the medical ward rounds in advance, so that they can prepare in advance, participate in the ward rounds on time, and ensure a quality of the medical ward rounds.

2400: Interaction and coordination of the medical ward rounds.

2500: Round a next patient, which including a plurality of steps as follow:

2510: the medical ward round accompanying robot arriving at a patient bedside in advance, informing the patient that a ward round is about to be carried out, and assisting the patient to prepare. If it is found that the patient leaves the ward due to a private trip and does not return, or the patient goes to a toilet and is expected to be unable to return to bed in a short period of time, which can be sent to the chief doctor and/or the chief nurse of the department to adjust the task planning, and a next patient can be rounded;

2520: if there is a shortage of materials, consumables and spare parts, which can be sent to the chief doctor and/or the chief nurse of the department in time to facilitate necessary supplementation;

2600: interactive collaboration after the medical ward rounds, including a data processing, an update of a medical record, a replenishment of items, and an assistance in an execution of the doctor order; and

2700: standby.

The medical ward round accompanying robot completes the task planning, returns to an indwelling area, implements cleaning, disinfect, recharge, and replenish necessary supplies and consumables, so as to prepare for a next medical ward round.

In a medical ward round process of an embodiment of the present invention, a medical ward round accompanying robot service process step 2400 comprises a plurality of steps as follow.

2410: A doctor and/or a nurse issuing an instruction.

The doctor and/or nurse can issue an instruction through a human-machine interaction interface, then the medical ward accompanying robot receives the instruction from the doctor and/or nurse, and arrives at a bedside of a specific patient in the department according to a preset planned route, stands close to the patient head, implements a biometric identification, and checks the name and bed number of the patient, etc.

2420: The medical ward round accompanying robot rendering a content.

The doctor and/or nurse can interact with a human-machine voice, a gesture or touch screen through a human-machine interaction interface, then a display of the medical ward round accompanying robot can display the patient medical data in real time.

2430: The medical ward round accompanying robot collecting a data.

The data can include an interactive data, a physical examination data, a physical and laboratory inspection data, a disease discussion data, a doctor order and/or a nurse order data. Wherein the interactive data includes an audio and/or video data of a doctor-patient communication when the doctor and/or nurse making a ward round. Wherein the physical examination data includes is an audio and/or video data of what the doctor and/or nurse see, hear, and ask when conducting the physical examination. Wherein the physical and laboratory inspection data includes the data of a physical inspection, an imaging inspection, a laboratory inspection, a wound inspection, and a drainage inspection when the doctor and/or nurse performing at the patient bedside. Wherein the disease discussion data refers to the data of an audio and/or video data during the disease discussion when the doctor and/or nurse analyze and discuss the patient condition at the bedside or at a discussion room. Wherein the doctor order and/or the nurse order data include a doctor and/or nurse voice order and/or an instruction data issued with a touch screen or a keyboard.

2440: The doctor and/or nurse signing a medical paperwork.

When a special explanation is required, such as an informed consent for a surgery, and/or an informed consent for an anesthesia, and/or an informed consent for a special examination, the doctor or nurse can issue an instruction through a voice or touch screen, then the medical ward round accompanying robot calls a medical document template and edits and revises the medical document template according to a content of the instruction. After the doctor or nurse reviews, revises and confirms, a paper version of the medical document is printed immediately when necessary for a signature, or the medical ward round accompanying robot displays an electronic version of the medical document for an electronic signature of both the doctor and/or the nurse and/or the patient.

2450: The medical ward round accompanying robot implementing a mission matter.

The doctor and/or the nurse can issue an instruction of a mission matter through the voice or touch screen, then the medical ward round accompanying robot broadcasts the mission matter by voice, and/or sends the mission matter to a mobile terminal of a patient or his/her guardians to achieve a purpose of a medical and health education. and

2460: The medical ward round accompanying robot storing data.

At an end of an on-site ward round of the patient, the medical ward round accompanying robot can generate a medical ward round data file of the patient to name, compress and store.

In a medical ward round link of an embodiment of the present invention, the medical ward round accompanying robot post medical ward round service process step 2600 comprises a plurality of step as follow:

2610: The medical ward round accompanying robot processing a data.

The medical ward round accompanying robot can extract a plurality of key feature value, such as a mental state, a sleep, a diet, a vital sign word, a drug name, a disease name, a surgery name, and a number and/or descriptive word, follow successively, and convert into a plurality of text file according to a specific logic algorithm, and list in a category of an inquiry, a physical examination, a physical inspection, a laboratory examination, a disease discussion, a doctor order, a nurse order, and an informed notice, etc., and transmit to the doctor and/or nurse through a communication network, load the patient electronic medical record file, and form a medical record according to a certain logical order. In addition, an original data such as an audio and/or video document of the patient ward round can be attached to a text log listed in the medical record.

For example, in a disease discussion session, when the medical ward round accompanying robot recognizes a voice communication content of the doctors in a medical team, the text log can be formatted as follows:
The doctor A: . . . [an original audio and/or video data]
The doctor B: . . . [an original audio and/or video data]
The doctor C: . . . [an original audio and/or video data]
The doctor A: . . . [another original audio and/or video data]

2620: The medical record being updated.

The doctor and/or nurse can refer to the medical record log, call an original data file of a current ward round when necessary, reproduce a scene of the medical ward round, assist the doctor and/or nurses to complete a editing and sorting of the medical record, and ensure that the medical ward round information is recorded in an electronic medical records instrument in a timely and accurate manner.

2630: The medical ward round accompanying robot replenishing an item.

At an end of the medical ward round, the medical ward round accompanying robot can have a self-check, including showing a power, an equipment working condition, and a remaining amount and difference of consumables, then prompt supplementation. and

2640: The medical ward round accompanying robot assisting in an execution of the doctor and/or nurse order.

After receiving the doctor and/or nurse order and performing a task, the medical ward round accompanying robot can prompt the doctor and/or nurse to prepare the medical equipment and consumables in advance according to the doctor and/or nurse order, carry out a special examination, and accompany the doctor and/or nurse to the ward with an item to provide an intravenous or intramuscular injection, an oral medication and/or other nursing care.

The medical ward round accompanying robot according to an embodiment of the present invention includes, but is not limited to, a human-machine interaction module, a data acquisition module, a data presentation module, a data processing module, a medical record log automatic generation module, a data security module, and a raw data document calling module.

In which, the human-machine interaction module can integrate multi-channel interaction methods, including a voice, a touch screen, an eyesight, an expression, an iris, a palm print, a handwriting, a gait, a gesture, a lip reading, a face, a DNA, and an idea, as well as a mobile smart terminal, a smart wearable device, and an implanted sensing chip in body, and realize the human-machine interaction.

In which, the data acquisition module comprises as follow:

(1) the data can be collected by the medical ward round accompanying robot with a microphone, a computer vision sensor, and an electrochemical sensor to obtain a raw data of the medical ward round;

(2) another medical robot, an intelligent bedside monitoring equipment, an intelligent patient wearable equipment, a smart home equipment, and an intelligent physical laboratory inspection equipment connected by the Internet of Things can obtain real-time a patient audio and/or video data, a monitoring data, a physical inspection data, and/or a laboratory inspection data;

(3) the human-machine interaction module obtains a remote interaction data between a guardian and the patient, and an interaction data between a medical care team and the patient; and

(4) a data on the execution of the doctor order and/or a nurse order is assisted by the medical ward round accompanying robot.

In which, the data presentation module refers to: a medical staff instructs the medical ward round accompanying robot through a human-machine interaction interface to display in real time on the touch screen of an electronic medical record, an imaging data, a pathology data, a gene map, a laboratory detection data, a human medical knowledge document and real-time retrieval data, an audio and/or video of a remote ward round, a medical examination data, and a ward round planning route.

In which, the data processing module can pack the data of the current ward round of the patient collected by the data acquisition module, create a folder, name the folder, compress and store the folder, form an original data file, and extract a feature value from the original data to form a text file in real time according to a logic algorithm.

In which, the medical record log automatic generation module is configured to list a text file formed by the data processing module in a chronological order accurate to a second, load an electronic medical record of the patient, and form a medical record log which can be attached on an original data document.

In which, the data security module can create a data center through a blockchain technology to store and distribute the patient medical record data, connect an IoT data, and upload a public and/or a private cloud server based entirely on an authorized blockchain smart contract. The patient data is written into a block in real time, and the doctor and/or nurse in the department can be authorized to call and read the patient medical record data.

A medical ward round accompanying robot according to an embodiment of the present invention can perform the medical ward round in cooperation with a medical staff and a machine. The medical ward round accompanying robot comprises an intelligent moving chassis and a robot body. The robot body at least comprises a power supply unit, a wireless communication unit, an equipment unit, a medicine and equipment unit, a cleaning and disinfection unit, a printing and copying unit, and a data storage and processing unit.

In which, the equipment unit includes but are not limited to an electrocardiograph instrument, an electroencephalograph instrument, an ultrasound instrument, a respiratory function tester instrument, a blood, urine and stool analyzer, and a plurality of conventional diagnosis and treatment instrument, the conventional diagnosis and treatment instrument includes but are not limited to a stethoscope, a percussion hammer, a flashlight, an otolaryngoscope, a vaginoscope, a pulse collector and a camera.

In which, the medical equipment unit includes but is not limited to a suture removal kit, a dressing kit, a first aid medicine, and a first aid equipment.

In which, the cleaning and disinfection unit includes but is not limited to a hand sanitizer, a disinfectant, a spray equipment, a sterilization towel, a medical glove, a medical mask, a medical hat, a medical goggle, and a medical isolation gown.

A system of an embodiment of the present invention comprises at least a mobile intelligent terminal, a cloud service, a 5G, a logistics system, an intelligent medical system, a human-machine fusion protocol, an intelligent wearable device, a VR/AR device, a map navigation system, an intelligent access control, and a plurality of software, the human-machine fusion protocol includes a communication protocol between the human and a general practitioner robot, the general practitioner robot and another robot and an intelligent hardware, the software includes a underlying operating system, an application program, and an algorithm.

A human-robot collaboration system of a medical service robot according to an embodiment of the present invention includes another medical robot connected with the Internet of Things, such as a surgical robot, a nursing robot, a sleeping care robot, a smart wearable device, a smart medical device, a memory, a processor, a storage, and a computer instruction running on the memory and on the processor. When the computer instruction is executed by the processor, the medical service robot can complete a human-machine cooperative operation.

A preferred embodiment of the present application provides a readable storage medium on which an instruction, a program, a code set or an instruction set are stored, and loaded and executed by a processor to implement a digital generation, a display and a usage of a medical record.

Of course, all or part of the processes in the methods of the above-mentioned embodiments can be completed by instructing a relevant hardware through a computer program, and the computer program can be stored in a non-volatile computer-readable storage medium. When the computer program is executed, the processes of the embodiments of the methods described above can be included. Any reference to the memory, the storage, the database or another medium used in the embodiments provided in this application can include a non-volatile and/or volatile memory.

Based on this, the control mode of the medical service robot can at least select a man-machine remote fusion mode, a man-machine fusion mode or an intelligent operation mode. In a case of the human-machine remote fusion mode, when the medical service robot operates autonomously, an operator can dynamically perceive its status in real time at a control terminal. Once a fault or error occurs, the operator can control or cut off its autonomous operation, provide a real-time help and optimize the operation. In a case of the human-machine fusion mode, the medical service robot performs an operation in collaboration with on-site a doctor, and/or a nurse, and/or a patient, and/or a patient escort. In a case of the intelligent operation mode, the medical service robot arrives at a scene and performs an operation completely autonomously.

The above are only the embodiments of the present invention, and do not limit a technical scope of the present invention. Therefore, any minor modification, equivalent change and modification made to the above embodiments according to a technical essence of the present invention still belong to the scope of the present invention, and within the scope of the technical solution. A Professional should appreciate that a skilled artisan may use different methods of implementing the described functionality for each particular application, but such implementations should not be considered beyond the scope of the present invention.

What is claimed is:

1. A medical service robot, being used to replace a doctor and/or a nurse in a medical service system, comprising:
a general practitioner robot configured to provide a medical service in an outpatient department;
a medical emergency robot configured to provide a medical emergency service in an emergency department; and
a medical ward round accompanying robot configured to provide an accompanying medical ward round service in an inpatient department.

2. The medical service robot according to claim 1, wherein the outpatient department comprises an outpatient clinic, an individual clinic, a health examination center, and/or a medical workstation; wherein the emergency department includes an emergency center, an emergency room, and/or an emergency workstation; and wherein the inpatient department includes an inpatient ward, an outpatient observation ward, and/or a family ward.

3. The medical service robot according to claim 1, wherein the general practitioner robot comprises a chassis and a robot body, the robot body comprises a display, a mechanical arm, a mechanical neck, and a main structure, the chassis configured to carry the robot body, the mechanical neck configured to connect the display and the main structure, the main structure comprises a medical care module, a specimen collection module, a specimen detection module, a physical inspection module, a daily care module, a central data processing module, a biosafety module, a data security module, and a power supply module.

4. The medical service robot according to claim 1, wherein the medical emergency robot comprises a chassis, a mechanical arm and a main structure, the chassis configured to carry the main structure and the mechanical arm, the mechanical arm is arranged on the main structure, the main structure comprises a state perception module, an environment perception module, a human-machine interactive module, a wireless communication module, a central data storage processing module, a physical inspection module, a laboratory detection module, a life support module, a surgical emergency module, a disinfection module, an intelligent storage module, a warning equipment, a universal bracket, and a display.

5. The medical service robot according to claim 1, wherein the medical ward round accompanying robot comprises a chassis and a robot body, the chassis configured to carry the robot body, the robot body comprises a power supply module, a wireless communication module, an equipment module, a medicine device module, a cleaning and disinfection module, a printing and copying module, a data storage processing module, and a human-machine collaborative module.

6. The medical service robot according to claim 3, wherein the mechanical arm comprises a left nine-axis mechanical arm and a right nine-axis mechanical arm, which are respectively connected with a left upper end of the main structure and a right upper end of the main structure.

7. The medical service robot according to claim 3, wherein the medical care module comprises a first box, the first box includes a blood pressure measurement instrument, a blood oxygen saturation measurement instrument, a pulse measurement instrument, a body temperature measurement instrument, and a grip strength measurement instrument, which is arranged on an outer wall of the main structure.

8. The medical service robot according to claim 3, wherein the specimen collection module comprises a finger blood collection instrument, a venous blood collection instrument, a pleural and ascites collection instrument, a urine collection instrument, a stool collection instrument, and a plurality of universal collection instrument, which are arranged on an upper part of the main structure; and wherein the specimen detection module includes a blood detection instrument, an excrement detection instrument, a urine detection instrument, a stool detection instrument, a pleural effusion and ascites detection instrument, an antigen antibody detection instrument, and a nucleic acid detection instrument, which is arranged inside the main structure.

9. The medical service robot according to claim 3, wherein the biosafety module comprises an internal disinfection device, an external disinfection device, an air purification device, and a waste treatment device; and wherein the data security module is configured to create a user data center, store and distribute the user data through a blockchain technology.

10. The medical service robot according to claim 3, wherein the physical inspection module comprises an electrocardiogram instrument, an electroencephalogram instrument, an X-ray inspection instrument, an ultrasound inspection instrument, and a respiratory function inspection instrument, which share the display and an intelligent cloud diagnosis workstation.

11. The medical service robot according to claim 4, wherein the chassis comprises a wheeled intelligent mobile structure, or a four-legged intelligent moving structure, or a crawler-type intelligent moving structure.

12. The medical service robot according to claim 4, wherein the mechanical arm comprises a grasping structure, a suction cup structure and an auxiliary diagnosis structure, the auxiliary diagnosis structure comprises a camera, an light, a 3D sensor, an intelligent stethoscope, and a sensor array.

13. The medical service robot according to claim 4, wherein the display comprises a touch screen, a detection device, a status perception module, a biometric identification module, an interactive module, a wireless communication module, and an air quality monitoring device.

14. The medical service robot according to claim 4, wherein the life support module comprises an oxygen generator, a negative pressure suction device, an endotracheal intubation device, a non-invasive ventilator, a defibrillator, a pacemaker, an automatic cardiopulmonary resuscitation machine and a first aid medicine; and wherein the surgical emergency module includes an external fixation splint, a plaster bandage, a tourniquet, a sterile dressing material, an ice pack, a disinfectant, and a folding stretcher, which are stored in an intelligent storage module.

15. The medical service robot according to claim 14, wherein the intelligent storage module comprises a small item storage part and a large item storage part, the small item storage part comprises a plurality of storage box, an induction conveyor belt, an induction door, a tray, and a plurality of storage cell, the storage box is arranged on both sides of the induction conveyor belt, and divided into a plurality of layer and area, the area includes a plurality of storage cell, the storage cell includes a runner instrument, the large item storage part comprises a plurality of chamber, the chamber comprises an induction hatch, a transmission board, a controller, a second micromotor, and a lead screw instrument.

16. A medical service method for replacing a doctor and/or a nurse to implement a medical service in a medical service system, comprising:
configuring an outpatient department by a general practitioner robot service process to replace the doctor/or nurse in an outpatient department;
configuring a medical emergency robot service process to replace the doctor/or nurse in an emergency department; and
configuring a medical ward round accompanying robot service process to replace the doctor/or nurse in an inpatient department.

17. The medical service method according to claim 16, wherein the general practitioner robot service process comprising:
deploying the general practitioner robot;
arriving at a predetermined service location by the general practitioner robot;
identifying a patient by the general practitioner robot;
communicating by the general practitioner robot with the patient with a help of a camera and an interactive module, and implementing a medical history collection and a physical examination;
implementing by the general practitioner robot a physical inspection, collecting a specimen and having a laboratory detection;
according to a program algorithm, confirming by the general practitioner robot a diagnosis and executing a doctor and/or nurse order;
integrating by the general practitioner robot a data of the medical history, the physical examination, the physical inspection, the laboratory detection, the doctor and/or nurse order and an execution of the doctor and/or nurse order to form a data set, and generate a medical record document; and
completing by the general practitioner robot a diagnosis and treatment operation.

18. The medical service method according to claim 16, wherein the medical emergency robot service process comprising:
assembling a plurality of function modules of the medical emergency robot according to a task;
having a self-check by the medical emergency robot;
monitoring environmental information by the medical emergency robot on standby or cruising at a predetermined location;
obtaining by the medical emergency robot an information and respondence to a need;
implementing the medical emergency robot a medical rescue;
having a self-check again, supplementing consumption, cleaning and disinfection by the medical emergency robot; and
continuing in a service by the medical emergency robot.

19. The medical service method according to claim 16, wherein the medical ward round accompanying robot service process comprising:
deploying the medical ward round accompanying robot;
having a human-machine collaborative training by the medical ward round accompanying robot and a medical staff;
preparation before a medical ward round;
performing an interaction and coordination of the medical ward round by the medical ward round accompanying robot;
making a next medical ward round by the medical ward round accompanying robot;
having an interactive collaboration after the medical ward rounds by the medical ward round accompanying robot; and
standbying for a next medical ward round by the medical ward round accompanying robot.

20. A medical service system, comprising:
a plurality of softwares, a plurality of algorithms, a cloud server, a 5G network, an intelligent medical system, a medical data encryption system, a diagnosis and treatment data recording system, a human-machine fusion protocol, a map navigation system, a pharmaceutical consumables and equipment update and replenishment system, a call transfer system, a logistics transportation system, an intelligent access control system and an expense settlement system configured to collaborate a general practitioner robot, a medical emergency robot and a medical ward round accompanying robot replacing a doctor and/or nurse to perform a medical service in a medical service system
